(19) United States
(12) Patent Application Publication
(10) Pub. No.: US 2010/0312174 A1
(43) Pub. Date: Dec. 9, 2010

(54) Peritoneal Dialysis System

(76) Inventor: Josef C. A. Hoffman, Irvine, CA (US)

Correspondence Address: Josef C.A. Hoffman, 189 Stonecliffe Aisle, Irvine, CA, 92603, US

(21) Appl. No.: 12/479,783

(22) Filed: Jun. 6, 2009

(51) Int. Cl. A61M 1/28

(52) U.S. Cl. 604/29

(57) ABSTRACT

The present invention is a sorbent-based portable peritoneal dialysis system that uses 2.5 liters of tap water per day. The system comprises a control unit, a sterilized disposable cassette, a sterilized disposable glucose solution cartridge and a sterilized sorbent cartridge, and a three liter removable fluid storage container. A supply of concentrated electrolytes solution and a venting sterilizing dialysate filter are contained in the cassette. The glucose and sorbent cartridges snap into the cassette, which snaps onto the control unit. The cartridges are replaced daily, and the cassette is replaced weekly. During use (typically while the patient sleeps at night), the system removes all spent dialysate from the patient every two hours. The system then returns two liters of regenerated, sterilized dialysate to the patient. The patient discards the spent dialysate in the morning.

### CROSS-REFERENCE TO RELATED APPLICATIONS

Two other associated utility patent applications were also electronically filed on this day: Jun. 5, 2009.

### BACKGROUND OF THE INVENTION

There are an estimated 600,000 dialysis patients in the United States in 2009. Approximately 60,000 of these patients use peritoneal dialysis, with the remainder using hemodialysis. The majority of peritoneal dialysis patients use Automated Peritoneal Dialysis (APD), which is typically conducted at night, while the patient is sleeping. In APD, an automated cycler exchanges spent dialysate in the patient's peritoneal cavity, with two liters of sterile, warmed fresh dialysate, completing four to six exchanges a night. Peritoneal dialysis patients who do not use APD, use Continuous Ambulatory Peritoneal Dialysis (CAPD), in which the patient manually exchanges two liters of dialysate per session, four to six times a day. These peritoneal dialysis methods have changed very little over the past 30 years.

In peritoneal dialysis, excess water, creatinine and urea (amongst other chemicals) are removed from the patient's bloodstream using the patient's peritoneal membrane as a filter membrane. Water diffuses from the bloodstream into the dialysate due to a relatively high concentration of glucose in the dialysate, which creates a tonicity gradient. Creatinine and urea also diffuse into the dialysate due to concentration gradients between the blood and the dialysate. The longer the dialysate is kept in a patient's peritoneal cavity, the less effective it becomes at removing excess water and toxins, because the chemical concentration gradients between the bloodstream and the dialysate approach equilibrium over time. Because of this, dialysate is typically kept in the patient's peritoneal cavity for about two hours at a time for patients with highly permeable peritoneal membranes, and about four hours at a time for patients with less permeable peritoneal membranes.

After the desired dialysate dwell time has elapsed, the spent dialysate is removed via the implanted single-lumen Tenckhoff catheter, and then discarded. Two liters of fresh, warmed, sterile dialysate is then instilled into the peritoneal cavity via the same catheter, and the above process is repeated. Typically, four to six dialysate exchanges are performed per day (or per night). Unless the dialysis patient gets a kidney transplant or switches to hemodialysis, this treatment must be performed every day, for the rest of the patient's life.

All existing methods of peritoneal dialysis include a number of drawbacks. First, it is very inconvenient for each peritoneal dialysis patient to receive, store, and man-handle up to 20 liters per day of fresh dialysate. The bags of dialysate are heavy, and they can take up to half a garage to store. Also, if a patient will be away from home, they must take a supply of 20 liters of dialysate per day, with them. Another drawback is that all existing peritoneal dialysates have a pH of approximately 5.4. This acidic solution irritates the peritoneal lining, causing many patients to permanently reject peritoneal dialysis after a few years.

Another drawback with all existing methods of peritoneal dialysis, is that all of the patient's proteins and amino acids that dissolve in the dialysate during treatment, are discarded with the spent dialysate. This leads to protein deficiency in some patients.

Another drawback with all existing methods of peritoneal dialysis, is that there are only three glucose concentrations commercially available for existing peritoneal dialysate. This is a drawback because some patients require concentrations below 1.5% or above 4.25%, in order to remove less or more water from their bodies than the current dialysates can remove. This occasionally includes patients that require dialysis and the concurrent addition of water to their bodies.

Another drawback with all existing methods of peritoneal dialysis, is that some dialysis patients have too low or too high levels of Sodium, Potassium, Magnesium, or Calcium in their bodies. All existing peritoneal dialysates offer only a single fixed concentration of these minerals. This requires the nephrologist to treat the imbalance using oral or injectable supplements, and/or special diets for the patient.

Another drawback with all existing methods of peritoneal dialysis, is that the fresh dialysate contains a number on non-biocompatible compounds, collectively known as Glucose Degradation Products (GDP's). While in the patient's peritoneal cavity, these molecules hasten the creation of another set of non-biocompatible compounds collectively known as Advanced Glycation Endproducts (AGE's). The present invention is designed to perform peritoneal dialysis with the lowest possible concentration of GDP's and AGE's, as well as eliminating all of the other drawbacks described above.

### BRIEF SUMMARY OF THE INVENTION

The portable peritoneal dialysis system of the present invention is a system and method for peritoneal dialysis that removes excess water, urea and creatinine from the patient. The system accomplishes this using 2.5 liters of warm tap water, fixed or unfixed urease, Zirconium-based cation and anion exchange chemicals, activated carbon, concentrated glucose solution, concentrated Calcium and Magnesium solution, a gas/liquid separator, and a sterilizing filter.

The system pumps 2 liters of dialysate into and out of the patient's peritoneal cavity in a “tidal” flow pattern. The dialysate is regenerated, degassed and sterile-filtered during each cycle. The system can perform dialysate exchanges as frequently as twice per hour, and it is designed to connect to a standard single-lumen implanted Tenckhoff catheter. Patients should use the system at least 8 hours per day. A carrying handle is included on the cassette and on the control unit, and the system is small and light enough to be easily carried if the patient is traveling or wishes to use it outside the home.

The complete system is comprised of a control unit, a three liter fluid storage container, a disposable cassette, a disposable sorbent cartridge, a disposable glucose cartridge, and a disposable venting sterilizing filter. The required type of electrolytes solution cartridge and the required glucose concentration in the dialysate are patient-specific, and they are prescribed/programmed by a nephrologist for each patient.

To use this system on the first day of the weekly use cycle, the patient first drains and discards all dialysate from his last daytime CAPD infusion (if any). The patient places the control unit on a night stand near his bed, and plugs it into a wall socket. He removes a sterilized cassette from its pouch and locks it onto the top of the control unit. He fills the dialysate storage container with 2.5 liters of warm tap water. He then removes a sterilized sorbent cartridge and a sterilized glucose solution cartridge from their pouches, and snaps them into their docking bays in the front face of the cassette. The patient removes the dialysate tube connector from its UV sterilizing port in the front panel of the control unit, connects the dialysate tube to his Tenckhoff catheter, pushes the “Start” button, and goes to sleep for the night. In the morning, he disconnects his Tenckhoff catheter, inserts the tube connector into its UV sterilizing port, and discards the spent dialysate in the fluid storage container.

To use this system on the following six nights of the weekly use cycle, the patient replaces the spent sorbent cartridge and the spent glucose solution cartridge, fills the dialysate storage container with 2.5 liters of warm tap water, then uses the system as described above.

This portable peritoneal system has seven advantages over existing APD and CAPD. The first advantage is the elimination of the need for 12 to 20 liters/day of dialysate to be delivered to the patient. This is achieved because the system creates dialysate as needed, using 2.5 liters of tap water once a night. This frees the patient from having to receive, store, and handle up to 20 liters (˜44 pounds) of fresh dialysate, and even more spent dialysate per day, for years. This system only requires storage space for, and handling of two small cartridges per day, and one cassette per week. This makes the system much easier to use when the patient is traveling.

The portable peritoneal system has a second advantage over existing APD and CAPD. Because the glucose in conventional peritoneal dialysate is more stable at a low (acidic) pH, commercial peritoneal dialysate is has a pH of approximately 5.4. This acidic pH irritates the peritoneal membrane, causing it to thicken and become increasingly less permeable. This, in turn, requires many peritoneal dialysis patients to switch to hemodialysis after a few years of peritoneal dialysis treatment. Because this system injects glucose into the dialysate at the time of use, rather than mixing and sterilizing them weeks or months in advance, the dialysate has an average physiological pH of approximately 7.2. The peritoneal membrane is less irritated by this solution, and this should help extend the number of years that PD patients can stay on peritoneal dialysis.

The portable peritoneal system has a third advantage over existing APD and CAPD. During peritoneal dialysis, natural proteins such as albumin diffuse into the dialysate. These proteins are important for maintaining good health and nutrition. Because existing peritoneal dialysis methods discard these proteins every day, approximately half of peritoneal dialysis patients suffer from malnutrition. Because this system regenerates and recycles the dialysate, most of these proteins are returned to the patient rather than being discarded. This reduces the chance of malnutrition in the patient.

The portable peritoneal system has a fourth advantage over existing APD and CAPD. In existing peritoneal dialysis, if the patient needs dialysis with a higher or lower glucose concentration, they must order, receive, and switch to bags containing the different glucose concentration. Only three concentrations (1.5%, 2.5%, and 4.25%) are commercially available. With this system, the glucose concentration in the dialysate can be easily and instantly changed on the system's control panel by the nephrologist, nurse, or technician. This allows the dialysate's glucose concentration to be quickly set to anything from 0.0% to 10.0%.

The portable peritoneal system has a fifth advantage over existing APD and CAPD. Setting the glucose concentration to 0.0% allows dialysis without the removal of any net water from the patient, in cases where the patient is dehydrated.

The portable peritoneal system has a sixth advantage over existing APD and CAPD. In existing peritoneal dialysis, a selection of formulations of dialysate are not commercially available. Whether a patient is acidotic, alkylotic, hypo or hyperkalemic, hypo or hypecalcemic, hypo or hypernatremic, they must use the same dialysate formulation. This system has a family of dialysate cartridges. Each is formulated to be appropriate for each patient's specific situation.

The portable peritoneal system has a seventh advantage over existing APD and CAPD. In existing APD cyclers, the instructions and alarms are, at best, readable messages on a screen. This system issues both verbal and readable instructions and/or alarm messages. Verbal messages are important because the system is meant to be used at night. When the system issues an alarm, the end user might be groggy or asleep, he will probably not be wearing his eyeglasses or contact lenses, and the screen might not be within his line of sight (when in bed). Also, many elderly patients respond better to verbal instructions and alarms, rather than readable instructions and alarms.

It is envisioned that the health and convenience advantages that this system enjoys over hemodialysis, APD and CAPD will encourage nephrologists and patients to transfer from those dialysis methods, to this system.

### BRIEF DESCRIPTION OF THE DRAWINGS

FIG. 1 depicts a front view of an embodiment of the control unit.

FIG. 2 depicts a top sectional view of an embodiment of the control unit.

FIG. 3 depicts a front view of an embodiment of the cassette

FIG. 4 depicts a top sectional view of an embodiment of the cassette.

FIG. 5 depicts a front view of an embodiment of the fluid storage container.

FIG. 6 depicts a side sectional view of an embodiment of the sorbent cartridge.

FIG. 7 depicts a side sectional view of an embodiment of the glucose solution cartridge.

FIG. 8 depicts a side sectional view of an embodiment of the venting sterilizing filter.

FIG. 9 depicts a top view of an embodiment of the venting sterilizing filter.

FIG. 10 depicts a front view of an embodiment of the complete portable dialysis system, including the control unit, the cassette, the sorbent cartridge, the glucose solution cartridge, and the venting sterilizing filter.

FIG. 11 depicts an electrical diagram for the control unit.

FIG. 12 depicts a dialysate flow diagram for the cassette.

### DETAILED DESCRIPTION OF THE INVENTION

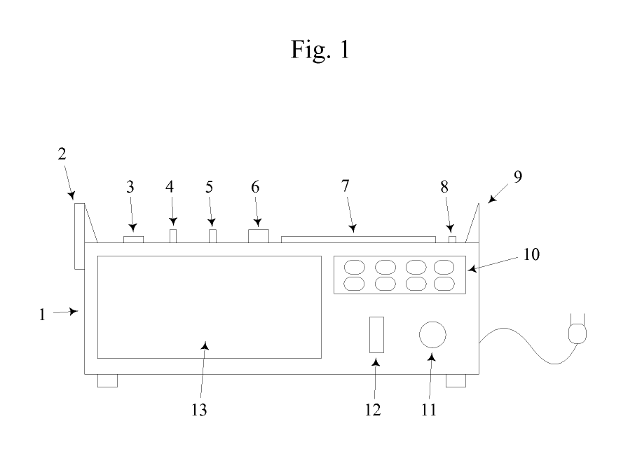

The Control Unit: As seen in FIG. 1, manual locking mechanism 2 is located at the top side of control unit 1. This mechanism keeps the cassette locked onto the control unit when the system is in operation, to prevent the patient from accidentally or intentionally separating the cassette from the control unit while the system is operating.

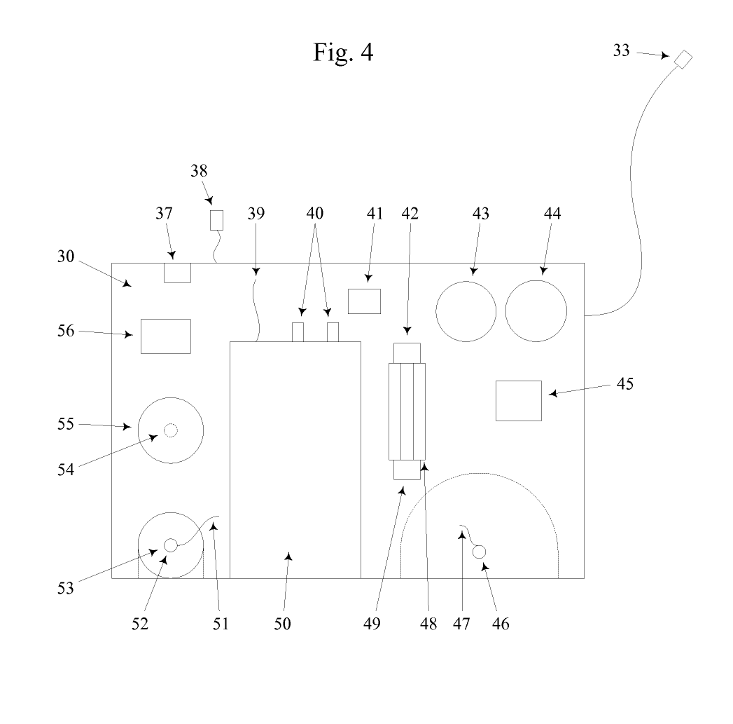

As seen in FIG. 1, electrical connector 3 is located in the top face of control unit 1. This connector mates with connector 56 in cassette 30 (see FIG. 4) when the cassette is attached to the control unit. This allows two-way electronic communication between the control boards in the control unit, and the sensors in the cassette.

As seen in FIG. 1, valve actuator 4 for the triple 3-way fluid valve is located in the top of control unit 1. This actuator turns the valve from position 1 to position 2, and back again. Dialysate is pumped from the patient when the valve is in position 1, and dialysate is pumped into the patient when the valve is in position 2.

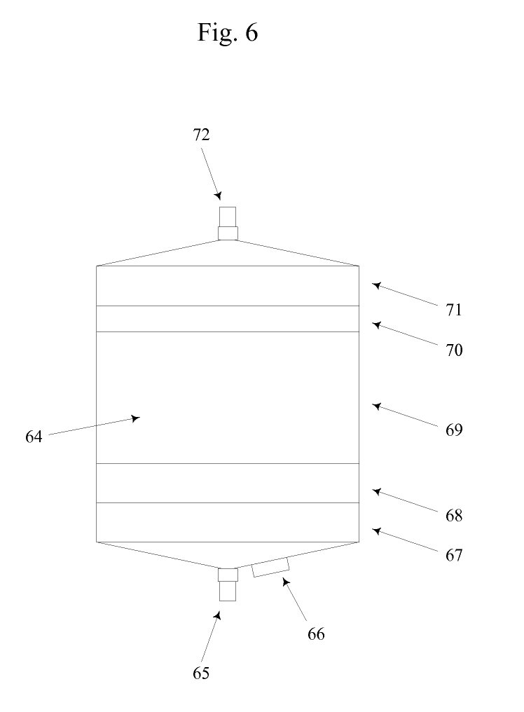

As seen in FIG. 1, valve actuator 5 for the air venting valve is located in the top of control unit 1. This actuator opens an air venting valve at the beginning of each evening's usage, to vent air that had been trapped in cassette 30 or in sorbent cartridge 64 (see FIG. 6).

As seen in FIG. 1, peristaltic pump rotor 6 is located in the top of control unit 1. This rotor pumps dialysate to and from the patient, and through the system. Peristaltic pump tubing assembly 48 in cassette 30 presses against the uppermost one-quarter arc of this rotor when the cassette is locked onto the control unit.

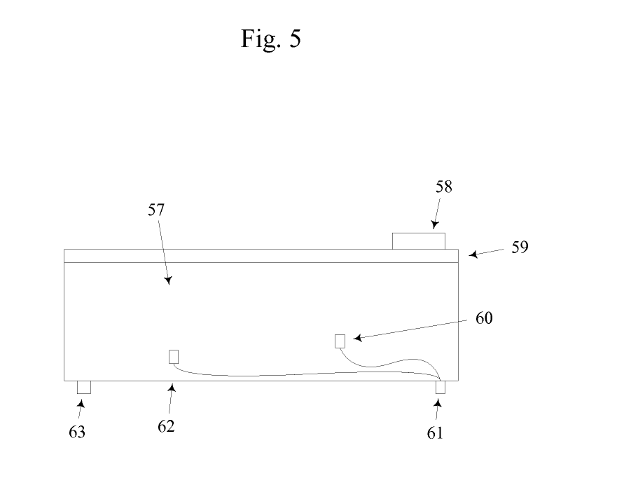

As seen in FIG. 1, heating pad 7 is located on the top of control unit 1. Fluid storage container 57 (see FIG. 5) sits directly on this heating pad during operation. The heating pad has a built-in thermostat that limits its upper temperature to approximately 130° F. during operation. It is also controlled by control board 18, which keeps the fluid in the fluid storage container at approximately 98.6° F. during operation. The heating pad uses 115V AC electrical power.

As seen in FIG. 1, electrical connector 8 is located at the top of control unit 1. This connector mates with electrical connector 61, that is built into the bottom of fluid storage container 57 (see FIG. 5). This allows communication between the temperature sensor and the fluid volume sensor that are built into the fluid storage container 57, and corresponding circuit boards 18 and 20 in control unit 1. The temperature probe can be an RTD, or a similar type temperature sensor. The fluid volume sensor can be a capacitance type sensor or an ultrasonic type sensor.

As seen in FIG. 1, cassette positioning guides 9 are located at the top left and tight sides of control unit 1. The guides precisely position the cassette as the user is attaching the cassette onto the control unit.

As seen in FIG. 1, control buttons 10 are located in the front panel of control unit 1. The control buttons might be a stainless steel membrane type. The control buttons include “On/Off”, “Start” and “Stop” buttons, buttons to increase or decrease the glucose concentration in the regenerated dialysate, and other control buttons.

As seen in FIG. 1, UV sterilizing port 11 is located in the front panel of control unit 1. When the system is not in use, the patient inserts dialysate tube connector 33 into this port. A sterilizing UV light inside the port turns on when the connector is inserted, and turns off automatically after about six minutes. The patient snaps a cap over the port's opening when the system is in use. If the connector is removed before the system is used again that evening, a verbal and readable alarm and instructional message will be issued.

As seen in FIG. 1, USB flash drive port 12, with a removable flash drive, is located in the front panel of control unit 1. Alarm incident data and a variety of operational data is downloaded to the flash drive, and the patient can bring the flash drive with him when he visits the nephrologist or the renal care nurse or technician.

As seen in FIG. 1, LCD screen 13 is located in the front panel of control unit 1. Alarm and instructional messages are displayed on this LCD screen, as needed. The LCD screen might be a back-lit, color, alphanumeric type.

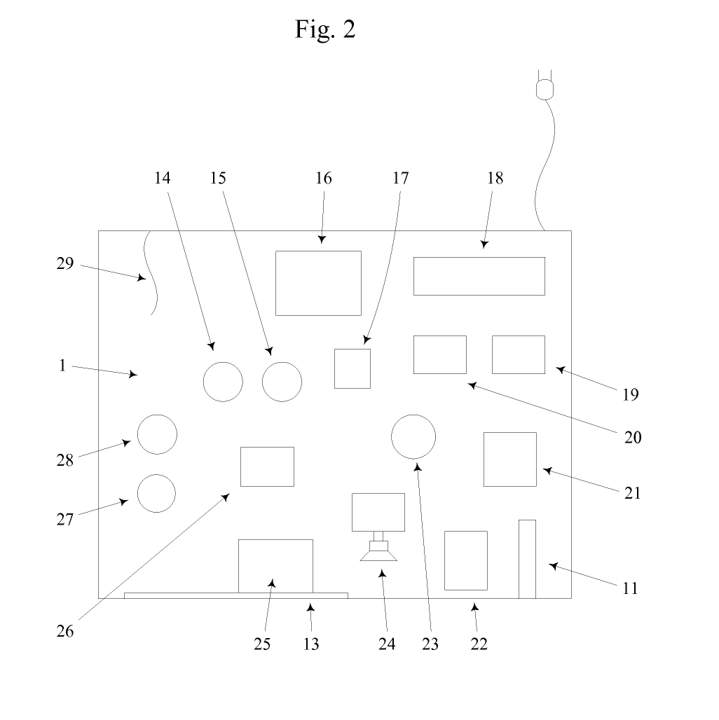

As seen in FIG. 2, gear motors 14 and 15 are mounted to the inside top of control unit 1. During operation, they turn triple 3-way fluid valve 43 and air venting valve 44 (both of which are in cassette 30) one-quarter turn clockwise or counter-clockwise, upon command from control board 18. The gear motors interface with the corresponding valves in the cassette, at the interface between control unit I and the cassette 30. The triple 3-way fluid valve will rotate to its “home” position before the cassette is separated from control unit. This will allow the valve to align with its actuator when that cassette, or a fresh cassette, is locked onto the control unit. Locating the gear motors in the control unit rather than in the cassette helps allow the cassette to be inexpensive enough to be disposable.

As seen in FIG. 2, power supply 16 is located in control unit 1. The power supply provides 12 Volt DC power to the various electronic components in the control unit. Fluid heating pad 1 is the exception, as it uses 115 Volt AC power.

As seen in FIG. 2, electronic two-axis inclinometer (tilt meter) 17 is located in control unit 1. The system must be kept fairly level during use. If the system is tilted excessively during use (such as being accidentally pulled off of the night stand), the system stops operating, and a verbal and readable alarm and instructional message is issued.

As seen in FIG. 2, control circuit board 18 is located in control unit 1. This board interfaces with the specialized circuit boards in the control unit, and it also monitors and/or controls each electronic component in control unit 1, cassette 30, disposable sterilizing filter 50, sorbent cartridge 64, and glucose solution cartridge 73. The hardware for this circuit board is commercially available, off-the-shelf.

As seen in FIG. 2, circuit board 19 is located in control unit 1. This circuit board electronically interfaces with the serial number chips it detects on cassette 30, disposable sterilizing filter 50, sorbent cartridge 64, and glucose solution cartridge 73. These disposable components contain a unique built-in serial number chip, which is read electronically when it touches a pair of lead wires. Whenever the control unit is turned on, or whenever a cassette, cartridge, or venting sterilizing filter is plugged in, the control unit automatically interrogates the serial number chip(s). The control unit remembers every serial number that is read, and compares every new serial number against those stored in its memory. If a serial number matches one already in memory, the system will stop, and a verbal and readable alarm and instructional message will be issued. This feature is to prevent a patient from reusing a used disposable component. RFID tags can be used instead of serial number chips. If RFID tags are used, an appropriate RFID circuit board would be included. The hardware for the serial number chips, or the RFID tags, are both commercially available, off-the-shelf.

As seen in FIG. 2, circuit board 20 for controlling the fluid volume sensor, is located in the control unit. This circuit board interfaces with fluid volume sensor 62, and calculates the volume of the fluid in fluid storage container 57. Fluid volume sensor 62 is either an RF capacitance probe or an ultrasonic probe. RF capacitance circuit boards and ultrasonic circuit boards for are both commercially available, off-the-shelf.

As seen in FIG. 2, LED/light meter 21 is located in control unit 1. This module focuses a tiny beam of light on colorimetric dissolved ammonia detector 45 (located in cassette 30), and measures the light that reflects off the detector. A decrease in the reflected light indicates that the dialysate contains dissolved ammonia. Ammonia is generated by the breakdown of urea in sorbent cartridge 64. When the sorbent cartridge becomes exhausted, it loses its ability to absorb the ammonia, and the ammonia is carried downstream in the dialysate. If any dissolved ammonia is detected during use, the system will immediately stop, and a verbal and readable alarm and instructional message will be issued.

As seen in FIG. 2, USB communication circuit board 22 is located in control unit 1. This board interfaces between a flash drive and control circuit board 18, which sends key alarm and operational data to the flash drive. This information can be downloaded as needed by a renal nurse or a nephrologist, typically once a month. The hardware for this circuit board is commercially available, off-the-shelf.

As seen in FIG. 2, gear motor 23 is located in control unit 1. Peristaltic pump rotor 6 is driven by this gear motor. The motor can be mounted vertically, using a 90° reducing gear box, or horizontally, using a straight reducing gear box. Control circuit board 18 keeps the motor at a constant rpm when it is running. Locating the gear motor in the control unit rather than in the cassette helps allow the cassette to be inexpensive enough to be disposable.

As seen in FIG. 2, sound generating circuit board and speaker 24 are located in control unit 1. This circuit board reproduces electronically stored verbal messages. The messages are situation-specific instructional and/or alarm messages, which are called up by control circuit board 18. The voice and language might be selectable between male and female, and English, Spanish, and possibly other languages. The hardware for this circuit board is commercially available, off-the-shelf.

As seen in FIG. 2, LCD screen circuit board 25 is located in control unit 1. This circuit board generates stored messages on LCD screen 13. The messages are situation-specific instructional and/or alarm messages, which are called up by control circuit board 18. The language might be selectable between English, Spanish, and possibly other languages. The hardware for this circuit board is commercially available, off-the-shelf.

As seen in FIG. 2, 115 Volt electrical relay 26 is located in control unit 1. This relay turns electrical power to heating pad 7 on and off, based on control input from control circuit board 18. Since this circuit board, and every other electrical component in the system, use 12 Volt DC power, a relay is necessary to control the 115 Volt power to the heating pad.

As seen in FIG. 2, solenoid actuator 27 is located in control unit 1. This actuator is for glucose solution cartridge piston pump 53, located in cassette 30. This actuator aligns with its pump at the interface between the control unit and the cassette. The pump's stroke frequency determines the concentration of glucose in the passing dialysate stream. Locating the solenoid actuator in the control unit rather than in the cassette helps allow the cassette to be inexpensive enough to be disposable.

As seen in FIG. 2, solenoid actuator 28 is located in control unit 1. This actuator is for electrolytes solution cartridge piston pump 54, located in cassette 30. This actuator aligns with its pump at the interface between the control unit and the cassette. The pump's stroke frequency determines the concentration of electrolytes in the passing dialysate stream. Locating the solenoid actuator in the control unit rather than in the cassette helps allow the cassette to be inexpensive enough to be disposable.

As seen in FIG. 2, lead wires 29 are located in control unit 1. These lead wires are for electronically interfacing with cassette serial number chip 37. These lead wires align with this chip when cassette 30 is locked onto control unit 1. The lead wires connect to circuit board 19, which electronically reads the unique serial number on the chip. Alternately, an RFID tag, antenna, and circuit board can be used instead of a serial number chip and circuit board.

The control unit tracks the number of hours the current cassette and cartridges have been used. If a cassette or a cartridge is about to be used beyond its lifespan, a verbal and readable alarm and instructional message is issued. The patient is instructed to immediately replace the cassette, cartridge, or filter with a new one.

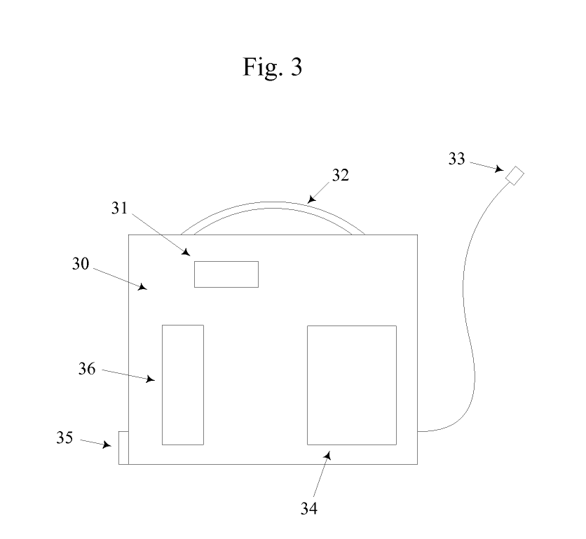

The Cassette: Cassette 30, as seen in FIG. 3, comes to the end user sterilized in a pouch. It is designed to lock onto control unit 1, and it is good for seven consecutive night's use. Venting sterilizing filter 50, sorbent cartridge 64, and glucose solution cartridge 73 all snap into docking bays in the front panel of the cassette. All of the wetted components in cassette 30 are biocompatible. Because it is disposable, the cassette is designed to be as inexpensive as possible.

As seen in FIG. 3, sterilizing filter docking bay 31 is located in cassette 30. Two self-sealing “break away” connectors 40 are located at the back of the bay, which mate with connectors 80 and 81 in the back face of venting sterilizing filter 50. The connectors are offset from the filter's centerline, making it impossible for the filter to be inserted upside down. Lead wires 39 are located in the back of the bay, for electronically reading serial number chip 83 that is built into the venting sterilizing filter. Alternately, an RFID tag can be used instead of a serial number chip.

As seen in FIG. 3, carrying handle 32 is located on the top of cassette 30. This enhances the portability of the cassette, after it has been removed from its pouch.

As seen in FIG. 3, dialysate tube with self-sealing connector 33 is located in cassette 30. This connector connects with the patient's tube dialysate during use, and it is inserted into UV sterilizing port 11 when the system is not in use.

As seen in FIG. 3, sorbent cartridge docking bay 34 is located in cassette 30. This bay has self-sealing “breakaway” connectors in its upper and lower surface. These connectors mate with connectors 65 and 72 at the top and bottom of sorbent cartridge 64. Lead wires 47 are located in the bottom surface of the bay, for electronically reading serial number chip 66 that is built into the sorbent cartridge. Alternately, an RFID tag can be used instead of a serial number chip.

As seen in FIG. 3, manual locking mechanism 35 is located at the bottom side of cassette 30. This mechanism keeps the cassette locked onto the control unit when the system is in operation, to prevent the patient from accidentally or intentionally separating the cassette from the control unit while the system is operating.

As seen in FIG. 3, glucose solution cartridge docking bay 36 is located in cassette 30. This bay has self-sealing “breakaway” connector 52 at its bottom. This connector mates with connector 77 at the bottom of glucose solution cartridge 73. Lead wires 51 are located in the bottom face of the bay, for electronically reading serial number chip 76 that is built into the glucose solution cartridge. Alternately, an RFID tag can be used instead of a serial number chip.

As seen in FIG. 4, electronic serial number chip 37 is located in the bottom rear face of cassette 30. This chip aligns with lead wires 29 at the top of control unit 1 when the cassette is locked onto the control unit. The chip contains a unique electronic serial number that identifies the specific cassette. Alternately, an RFID tag can be used instead of a serial number chip.

As seen in FIG. 4, tube with self-sealing “break away” connector 38 is located in cassette 30. This connector connects with fluid storage container connector 63, and it allows fluid to flow between the fluid storage container and the cassette.

As seen in FIG. 4, lead wires 39 are located at the rear face of venting sterilizing filter docking bay 31, in cassette 30. The lead wires align with serial number chip 83 on venting sterilizing filter 50, when a filter is snapped into the bay. The lead wires allow the chip's unique serial number to be read by circuit board 19 in control unit 1. Alternately, an RFID tag and circuit board can be used instead of a serial number chip and circuit board.

As seen in FIG. 4, two self-sealing “break away” connectors 40 are located in the back face of filter docking bay 31, in cassette 30. These connectors mate with connectors 80 and 81 in the back face of venting sterilizing filter 50. The connectors are offset from the filter's centerline, making it impossible to insert the filter upside down.

As seen in FIG. 4, fluid pressure sensor 41 is located in cassette 30, just upstream of the venting sterilizing filter. It continuously monitors the dialysate pressure at that location. An excessively low dialysate pressure at this location probably indicates a pinhole or a tear in the filter membrane. An excessively high dialysate pressure at this location probably indicates that the filter is becoming clogged. In either case, the system would turn off and a verbal and readable alarm and instructional message is issued. The patient would pull the “problem” filter out of the cassette, remove a replacement filter from its sterile pouch, plug the new filter into the cassette, and press the “Start” button. The system remembers where it was in the nightly use cycle, and it continues operating from that point. It might occasionally be necessary to replace a venting sterilizing filter with a fresh one before an entire week has passed, due to clogging from fibrin or other biological debris.

As seen in FIG. 4, fluid pressure sensor 42 is located in cassette 30, just upstream of peristaltic pump fixture 48. This pressure sensor is primarily used to detect a vacuum spike as dialysate is being pumped out of the patient's peritoneal cavity. A vacuum spike at this location indicates that all spent dialysate has been emptied from the cavity.

As seen in FIG. 4, triple 3-way fluid valve 43 is located in cassette 30. This quarter-turn valve routes dialysate to or from the patient, and to or from fluid storage container 57, as well as through the cassette.

As seen in FIG. 4, air venting valve 44 is located in cassette 30. This valve opens for about 20 seconds at the beginning of the first regeneration cycle of each evening's use, to vent air that was trapped in sorbent cartridge 64.

As seen in FIG. 4, colorimetric dissolved ammonia sensor 45 is located in cassette 30, just downstream of sorbent cartridge 64. This sensor has a hydrophobic membrane that is in contact with the dialysate, with chemicals impregnated on the outer face that change color in the presence of dissolved ammonia. When the cassette is locked onto control unit 1, this sensor aligns with light source and light meter 21 that are located in the control unit. The light meter detects the color change, and sends a signal to control circuit board 18.

As seen in FIG. 4, two self-sealing “break away” connectors 46 are located in the upper and lower surfaces of sorbent cartridge docking bay 34, in cassette 30. These connectors mate with connectors 65 and 72, located at the top and bottom of sorbent cartridge 64.

As seen in FIG. 4, lead wires 47 are located in the bottom surface of the bay, in cassette 30. The lead wires align with electronic serial number chip 66 that is built into sorbent cartridge 64, when a sorbent cartridge is snapped into docking bay 34. The lead wires allow the chip's unique serial number to be read by circuit board 19 in control unit 1. Alternately, an RFID tag and circuit board can be used instead of a serial number chip and circuit board.

As seen in FIG. 4, a short section of peristaltic pump tubing, set in semi-circular support fixture 48, is located in the bottom face of cassette 30. This flexible and durable section of tubing is maintained in a semi-circular shape by the semi-circular fixture. When the cassette is locked onto the control unit, this tubing is pressed against peristaltic pump rotor 6, which is located in the top of control unit 1.

As seen in FIG. 4, fluid pressure sensor 49 is located in cassette 30, just downstream of peristaltic pump fixture 48. During operation, a low pressure at this location indicates a dialysate leak somewhere in the cassette, and a high pressure at this location indicates a dialysate blockage somewhere in the cassette. If either situation happens, a verbal and readable alarm and instructional message is issued.

As seen in FIG. 4, venting sterilizing filter 50 is located in filter docking bay 31, in cassette 30. This filter comes installed in every new cassette. “Extra” filters are also packaged by themselves in pouches, then sterilized. During use, if an alarm message instructs the user to replace the filter, the user merely slides the old filter out of its docking bay, then slides the fresh filter into the bay until the two self-sealing connector pairs snap together.

As seen in FIG. 4, lead wires 51 are located in the lower surface of glucose solution cartridge docking bay 36, in cassette 30. The lead wires align with electronic serial number chip 76, which is built into glucose solution cartridge 73, when a glucose solution cartridge is snapped into its docking bay. The lead wires allow the chip's unique serial number to be read by circuit board 19 in control unit 1. Alternately, an RFID tag and circuit board can be used instead of a serial number chip and circuit board.

As seen in FIG. 4, self-sealing “break away” connector 52 is located in the lower surface of glucose solution cartridge docking bay 36, in cassette 30. This connector mates with self-sealing “break away” connector 77, located at the bottom of glucose solution cartridge 73.

As seen in FIG. 4, self-priming piston pump 53 is located in cassette 30. This pump pumps 100 μl/stroke of glucose solution into the passing dialysate stream. The pump is actuated by solenoid actuator 27, which is located in control unit 1, directly below this pump when the cassette is locked onto the control unit.

As seen in FIG. 4, self-priming piston pump 54 is located in cassette 30. This pump pumps 10 μl/stroke of electrolytes solution into the passing dialysate stream. The pump is actuated by solenoid actuator 28, which is located in control unit 1, directly below this pump when the cassette is locked onto the control unit.

As seen in FIG. 4, electrolytes solution cartridge 55 is located in cassette 30. It is permanently attached to piston pump 54, and it contains enough electrolytes solution for one week's use. The cartridge has a biocompatible internal bladder that slowly collapses as solution is pumped out of it. This bladder is highly impermeable to air and water vapor.

As seen in FIG. 4, electrical connector 56 is located in the bottom surface of cassette 30. This connector mates with connector 3, located in the top surface of control unit 1, when the cassette is locked onto the control unit. This connector allows electronic communication between the sensors in the cassette and the circuit boards in the control unit.

The cassette must be replaced about once per week, for three reasons. First, the built-in electrolytes solution cartridge in the cassette will run out of electrolytes solution. Second, a biofilm will gradually coat the internal wetted surfaces of the valves, the tubing, and the venting sterilizing filter. And finally, microbes on the interior wetted surfaces will excrete gradually increasing level of endotoxins into the passing dialysate.

The Fluid Storage Container: As seen in FIG. 5, three liter fluid storage container 57 snaps onto the top of control unit 1. It is removable, and it holds warm tap water at the beginning of the first cycle of each evening, and warm spent dialysate thereafter. Each morning, the user empties it, and rinses it out.

As seen in FIG. 5, filling cap and port 58 is located in lid 59 of fluid storage container 57. This port is used to fill the fluid storage container with 2.5 liters of warm tap water each evening, immediately before use.

As seen in FIG. 5, lid 59 is located on fluid storage container 57. The lid snaps onto the fluid storage container, and it is removable. Removing it allows the interior of the fluid storage container to be rinsed out or washed.

As seen in FIG. 5, fluid temperature sensor 60 is built into the wall of fluid storage container 57. It is wired to electrical connector 61. During use, the control unit continually monitors the temperature of the tap water or spent dialysate in the storage container. If the temperature is higher or lower than approximately 98.6° F., the system either allows the liquid to cool down, or it heats it up, as necessary.

As seen in FIG. 5, electrical connector 61 is built into the bottom of fluid storage container 57. This connector mates with electrical connector 8 that is in the top of control unit 1. This allows electronic communication between the temperature sensor and the fluid volume sensor that are built into the fluid storage container 57, and corresponding circuit boards 18 and 20 in control unit 1.

As seen in FIG. 5, fluid volume sensor 62 is built into fluid storage container 57. This sensor can be an RF capacitance probe or an ultrasonic transducer, and it continuously measures the volume of fluid in the container. It is wired to electrical connector 61.

As seen in FIG. 5, self-sealing “break away” connector 63 is located in the bottom of fluid storage container 57. This connector mates with self-sealing “break away” connector 38, located at the bottom rear of cassette 30. It allows fluid to flow between the fluid storage container and the cassette.

The Sorbent Cartridge: Sorbent cartridge 64 (as seen in FIG. 6) is good for up to 12 hours of continuous use, and the patient must install a fresh cartridge each evening. During each regeneration cycle the sorbent cartridge removes all electrolytes except Na⁺ from the dialysate. The sorbent cartridge removes most of the glucose from the dialysate on the first regeneration cycle, but very little glucose on subsequent regeneration cycles. To prevent a patient from installing the sorbent cartridge upside down, a self-sealing “break away” male connector is at one end, and a self-sealing “break away” female connector is at the other end. Every sorbent cartridge is sealed in a Tyvek/poly pouch before being sterilized using Gamma radiation.

As seen in FIG. 6, self-sealing “break away” inlet connector 65 is located at the bottom of sorbent cartridge 64. When the sorbent cartridge is snapped into docking bay 34 in cassette 30, this connector mates with lower self-sealing “break away” connector 46.

As seen in FIG. 6, electronic serial number chip 66 is located at the bottom of sorbent cartridge 64. This chip aligns with lead wires 47 in the bottom surface of sorbent cartridge docking bay 34, when the cassette is locked onto the control unit. The chip contains a unique electronic serial number that identifies the specific sorbent cartridge. Alternately, an RFID tag can be used instead of a serial number chip.

As seen in FIG. 6, the sorbent cartridge contains five chemical layers, which are (from bottom to top): about 45 g of activated carbon 67 (available from many chemical suppliers), about 30 g of urease 68 (available from many chemical suppliers) that is bonded (fixed) in a mono-, bi-, or tri-layer onto inert particles, about 380 g of the Na⁺ and H⁺ form of Zirconium Phosphate 69 (available from MEL Chemicals, Inc.), about 35 g of Hydrous Zirconium Oxide 70 (available from MEL Chemicals, Inc.), and about 45 g activated carbon 71. The cartridge is packaged in a pouch and sterilized with ˜30 kGy of Gamma radiation. Alternately, the urease can also be unfixed, and blended with powdered Alumina. Each chemical layer in the cartridge is stable after Gamma sterilization and at least 6 months storage at 30° C.

As shown in FIG. 6, activated carbon layers 67 and 71 absorb creatinine, uric acid, heavy metals, chloramines, some “middle molecules”, and miscellaneous organic molecules. They do not add any chemicals to the dialysate stream, and they do not affect the pH of the dialysate. Activated carbon is included as the first layer in the cartridge because some metal ions that might be in the dialysate can deactivate urease layer 68, so the activated carbon must absorb these ions before the dialysate reaches the urease layer.

As seen in FIG. 6, urease layer 68 might be chemically bonded (fixed) onto particles of an inert substrate in mono-, bi- and tri-layer thicknesses (known as fixing). Approximately 40% of the urease's original bioactivity is lost due to the fixing and Gamma sterilization processes. Unfixed urease or powdered jack bean meal mixed with powdered Alumina might be used instead of fixed urease. The urease breaks the incoming urea into ammonia, ammonium, carbon dioxide, OH⁻, and bicarbonate.

As seen in FIG. 6, Zirconium Phosphate layer 69 serves as a cation exchanger, giving up H⁺ and Na⁺ for ammonium, K⁺, Ca⁺², Mg⁺², and other incoming cations. The Zirconium Phosphate donates Na⁺ if its pH is above that of the dialysate, and it donates H⁺ if its pH is below that of the dialysate. To accomplish this, the pH of fresh Zirconium Phosphate in water is controlled to be about 6.2, but this can be varied. The amount of Zirconium Phosphate required is determined by the amount of urea a large patient would generate in 24 hours (which can be as high as 20 grams), plus a safety factor.

As seen in FIG. 6, Hydrous Zirconium Oxide layer 70 is amphoteric, which means it acts as an anion exchanger if its pH is above the dialysate pH, it acts as a mixed ion exchanger if its pH is roughly equal to the dialysate pH, and it acts as a cation exchanger if its pH is below the dialysate pH. In this system, Hydrous Zirconium Oxide (possibly mixed with Zirconium Carbonate) serves two purposes. First, it acts as an anion exchanger, giving up acetate or bicarbonate for incoming phosphate, fluoride, and heavy metals. Second, it controls the pH of the dialysate that is exiting the sorbent cartridge. The system maintains the pH of the regenerated dialysate to an average of about 7.2 over the lifespan of the sorbent cartridge. To accomplish this, the pH of fresh Hydrous Zirconium Oxide is controlled to be about 8.0, but this can be varied.

As seen in FIG. 6, uppermost activated carbon layer 71 is the final layer in the sorbent cartridge. It absorbs any non-biocompatible chemicals that might have come from the sorbent cartridge chemicals themselves. A filter is included at the exit from the cartridge, to prevent any sorbent chemical particles from being carried downstream with the dialysate.

As seen in FIG. 6, self-sealing “break away” outlet connector 72 is located at the top of sorbent cartridge 64. This connector mates with upper self-sealing “break away” connector 46 when a sorbent cartridge is snapped into docking bay 34 in cassette 30.

The Electrolytes Solution and Glucose Solution Cartridges: Electrolytes solution cartridge 55 has a capacity for one week's use. Every few seconds during each dialysate regeneration cycle, a pump pumps a some concentrated Calcium Acetate/Magnesium Acetate solution (or concentrated Calcium Bicarbonate/Magnesium Bicarbonate solution), into the dialysate stream that has passed through the sorbent cartridge. This is necessary because the sorbent cartridge removes all Ca⁺² and Mg⁺² ions from dialysate that passes through it. The pump stroke volume is 10 μl. The pump's stroke frequency is controlled to keep the electrolyte concentration in the treated dialysate at the correct physiological level.

Since the cassette (and the built-in electrolyte solution cartridge) is sterilized with ˜30 kGy of Gamma radiation, the electrolyte solution must be chemically stable after Gamma sterilization and at least 6 months storage at 30° C. The cartridge containing the concentrated electrolyte solution contains very little trapped air, because any bubbles would be pumped as if they were electrolytes solution, which would result in an under-concentration of electrolytes in the treated dialysate. The cartridge has a collapsible inner pouch, which is highly impermeable to air and water vapor over its shelf life. This is required because evaporation of water from the electrolytes solution would result in over-concentration of electrolytes in the treated dialysate.

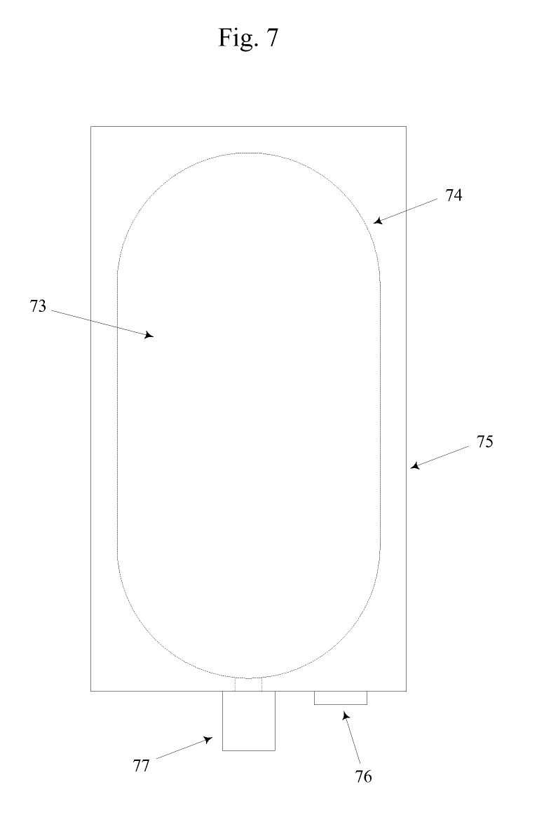

Glucose solution cartridge 73 is shown in FIG. 7. It is good for up to 12 hours of continuous use, so the patient must install a fresh cartridge each evening. The glucose solution cartridge contains from 100 ml to 250 ml of near-saturated sterile glucose solution, at an acidic pH. Every few seconds during the dialysate regeneration cycle, a pump pumps some concentrated glucose solution into the passing dialysate stream. This is necessary because the patient's body slowly absorbs glucose from the dialysate in his peritoneal cavity. The pump stroke volume is about 100 μl. The pump's stroke frequency is controlled to keep the glucose concentration in the treated dialysate at the desired level. Since the cartridge is sterilized with ˜30 kGy of Gamma radiation, the glucose solution must be chemically stable after Gamma sterilization and at least 6 months storage at 30° C.

The standard concentration of glucose in PD dialysate is 1.5%, but the device can to controlled to make the glucose concentration higher or lower. Dialysate glucose concentrations from 0.0% to 10% are possible. For many peritoneal dialysis patients, the amount of excess water to be removed varies over time. In order to accommodate this, the dialysate's glucose concentration can be increased or decreased via the control panel. A higher dialysate glucose concentration will remove more excess water from the patient, and a lower dialysate glucose concentration will remove less excess water.

The cartridge containing the concentrated glucose solution contains very little trapped air, because any bubbles would be pumped as if they were glucose solution, which would result in an under-concentration of glucose in the treated dialysate. The cartridge has a collapsible inner pouch, which is highly impermeable to air and water vapor over its shelf life. This is required because evaporation of water from the glucose solution would result in over-concentration of glucose in the treated dialysate. Every glucose solution cartridge is sealed inside a Tyvek/poly pouch before Gamma sterilization.

Traditional peritoneal dialysis typically results in about 8 grams of suspended proteins and amino acids being discarded per day, which can cause protein anemia in the patient. This device discards less than half of the proteins and amino acids that become suspended in the dialysate, and instead returns most of them to the patient. This results in a reduced loss of proteins loss by the patient.

As seen in FIG. 7, cartridge bladder 74 is located inside the casing of glucose solution cartridge 73. Its volume is from 100 ml to 250 ml, which is enough glucose solution for one day's use. The bladder is biocompatible, and it slowly collapses as solution is pumped out of it. The bladder is highly impermeable to air and water vapor.

As seen in FIG. 7, cartridge casing 75 is the outermost element of glucose solution cartridge 73. The casing provides protection and support for internal cartridge bladder 74. The casing has a tiny hole in the end opposite the connector, which allows air to ingress as the bladder slowly collapses as solution is pumped out.

As seen in FIG. 7, electronic serial number chip 76 is located at the bottom of glucose solution cartridge 73. This chip aligns with lead wires 51 in the bottom surface of glucose solution cartridge docking bay 36, when a glucose solution cartridge is snapped into the docking bay. The chip contains a unique electronic serial number that identifies the specific sorbent cartridge. Alternately, an RFID tag can be used instead of a serial number chip.

As seen in FIG. 7, self-sealing “break away” connector 77 is located at the bottom of glucose solution cartridge 73. When the glucose solution cartridge is snapped into docking bay 36 in cassette 30, this connector mates with self-sealing “break away” connector 52.

The Venting Sterilizing Filter: Gaseous carbon dioxide is one of the chemicals generated in the sorbent cartridge's urease layer. Because it is undesirable to introduce any gas into the patient's peritoneal cavity, the CO₂, and any air bubbles in the sorbent cartridge or the cassette tubing, are vented by air venting valve 44 and venting sterilizing filter 50. The venting sterilizing filters combine a large membrane surface area with a minimal internal volume. This creates the minimum possible back pressure in the dialysate, while having the minimum internal volume from which air must be purged. The filter includes hydrophobic membrane 78 with an average pore diameter of 0.2μ, and parallel hydrophilic membrane 79, also with an average pore diameter of 0.2μ. The filter is held in a horizontal orientation when it is installed in docking bay 31, in cassette 30.

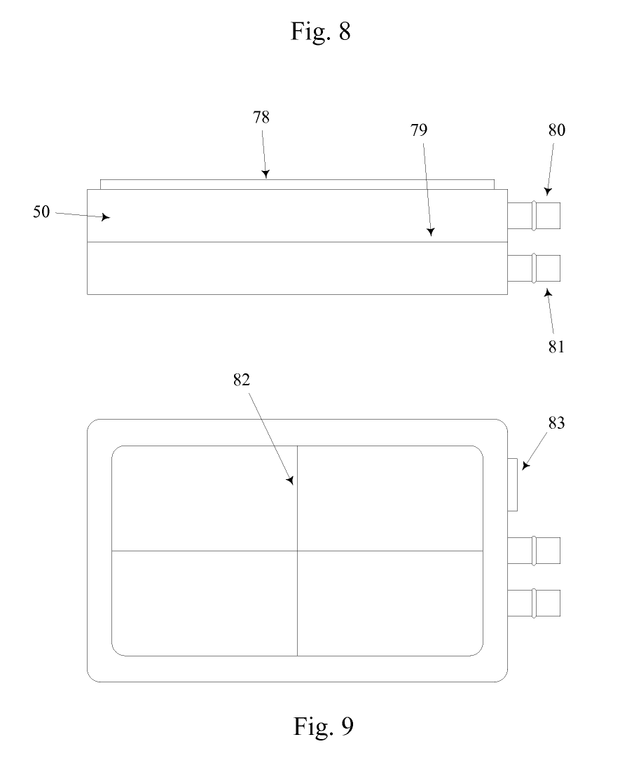

As seen in FIG. 8, hydrophobic membrane 78 will allow gases to pass through, but not dialysate. Hydrophilic membrane 79 will allow dialysate to pass through, but not gasses. Thus, this filter acts as both a dialysate sterilizer and as a gas/liquid separator, removing any entrained air or CO₂ from the dialysate. Venting entrained gas is particularly important after a fresh sorbent cartridge is snapped into a cassette, and/or after a fresh cassette is snapped onto a control unit, because they both contain a quantity of trapped air that must be vented.

As seen in FIG. 8, self-sealing “break away” inlet connector 80 and self-sealing “break away” outlet connector 81 are located in the rear surface of venting sterilizing filter 50. When a filter is slid into filter docking bay 31, these connectors mate with self-sealing “break away” connectors 40, located in the back face of the docking bay. The connectors are positioned offset from the center line of the filter, making it impossible for the filter to be inserted upside down.

As seen in FIG. 9, support bars 82 are located on the outer surface of hydrophobic filter 78, on venting sterilizing filter 50. These bars provide physical support to the filter membrane, against the fluid pressure inside the filter when the system is in use.

As seen in FIG. 9, electronic serial number chip 83 is located on the back surface of venting sterilizing filter 50. When a filter is slid onto filter docking bay 31, this chip aligns with lead wires 39 in the back surface of sterilizing filter docking bay. The chip contains a unique electronic serial number that identifies the specific filter. Alternately, an RFID tag can be used instead of a serial number chip.

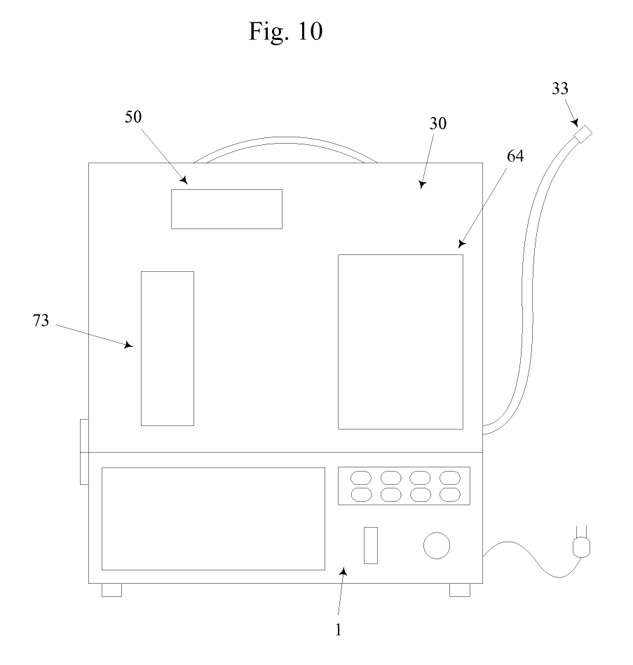

Peritoneal Dialysis System: FIG. 10 illustrates the entire peritoneal dialysis system, as it would appear during use. Cassette 30 is locked onto the top of control unit 1. Venting sterilizing filter 50 is snapped into its docking bay, sorbent cartridge 64 is snapped into its docking bay, and glucose solution cartridge 73 is snapped into its docking bay. Dialysate tube connector 33 would be connected to the patient's Tenckhoff catheter during dialysis. Fluid storage container 57 can not be seen in this view because it is located directly behind cassette 30, on top of control unit 1.

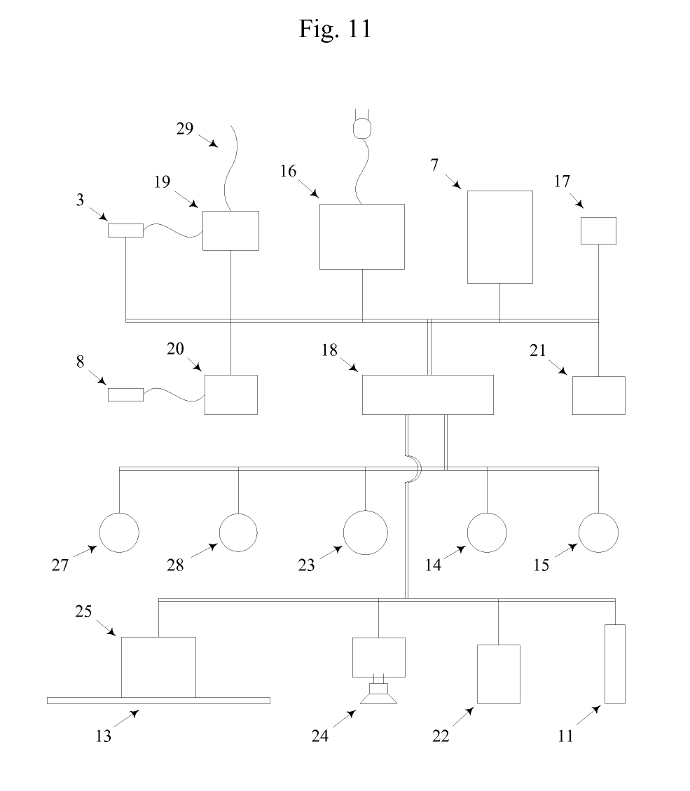

Electrical Diagram: FIG. 11 illustrates the electrical connections between the various electrical and electronic components in control unit 1. Power supply 16 sends 12 Volt DC and 115 Volt AC electrical power to control circuit board 18, and every component in the control unit communicates with, and receives electrical power via, the control circuit board. The components are connected to the control circuit board via a power/communications bus. Electrical connector 8 connects the temperature sensor and the fluid volume sensor in the fluid storage container, to circuit board 20. Electrical connector 3 connects lead wires 39, 47 and 51 in cassette 30, to circuit board 19. Circuit board 19 also has lead wires connected directly to it, for reading cassette serial number chip 37. Electrical connector 3 also connects fluid pressure sensors 41, 42 and 49 to control circuit board 18.

As seen in FIG. 11, the following components are connected to control circuit board 18, via a power/communications bus: electrical connector 3, serial number (or RFID) circuit board 19, power supply 16, heating pad 7, inclinometer 17, fluid volume circuit board 20, dissolved ammonia sensor light and light meter 21, glucose solution solenoid pump 27, electrolytes solution solenoid pump 28, peristaltic pump gear motor 23, triple 3-way valve gear motor 14, venting valve gear motor 15, LCD screen circuit board 25, voice circuit board and speaker 24, USB communication circuit board 22, and UV sterilizing port 11.

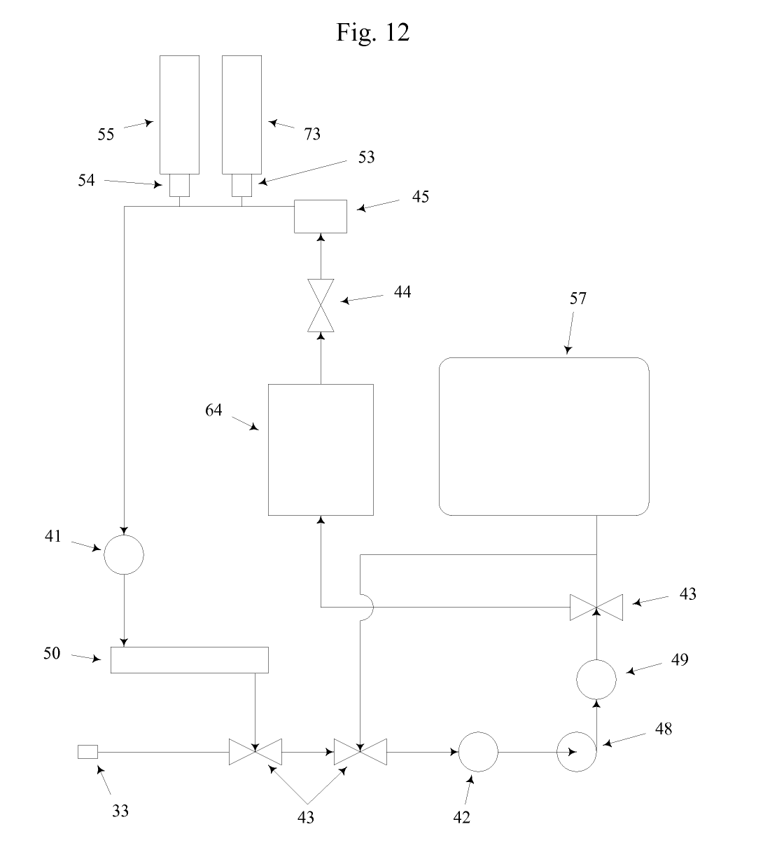

Dialysate Flow Diagram: FIG. 12 illustrates the dialysate flow paths in cassette 30. When spent dialysate is being pumped out of the patient's peritoneal cavity, it first passes through dialysate tube and connector 33, then through the upper and middle sections of triple 3-way valve 43, through pressure sensor 42, on its way to peristaltic pump assembly 48. The pump pumps the spent dialysate through pressure sensor 49, through the lower section of triple 3-way valve 43, and into fluid storage container 57.

When the spent dialysate is to be regenerated and pumped into the patient, triple 3-way valve 43 rotates 90° to position 2. The spent dialysate is then pumped from fluid storage container 57, through the middle section of triple 3-way valve 43, through pressure sensor 42, to peristaltic pump assembly 48. The pump pumps the spent dialysate through pressure sensor 49, through the lower section of triple 3-way valve 43, and through sorbent cartridge 64. The purified solution exits the sorbent cartridge, and flows through venting valve 44 and dissolved ammonia sensor 45, then past glucose solution pump 53 and electrolytes solution pump 54. The fully regenerated dialysate then flows through pressure sensor 41, venting sterilizing filter 50, the upper section of triple 3-way valve 43, dialysate tube and connector 33, and back into the patient's peritoneal cavity.

The Operating Cycle: Dialysate is pumped from the patient's peritoneal cavity at about 130 ml/minute (or perhaps a different flow rate) by peristaltic pump rotor 6 in control unit 1. The dialysate flow rate is monitored indirectly, by monitoring and controlling the pump's rpm. The incoming spent dialysate is pumped into fluid storage container 57.

When fluid pressure sensor 42 (just upstream of the dialysate pump) senses a sudden vacuum, the patient's peritoneal cavity is considered to be empty. Dialysate pump rotor 6 then stops, triple 3-way fluid valve 43 rotates to position #2, and dialysate pump rotor 6 restarts. Two liters of warm, spent dialysate is then pumped from fluid storage container 57 at 130 ml/minute (or perhaps a different flow rate), through sorbent cartridge 64, through dissolved ammonia detector 45, past glucose solution pump 53 and electrolytes solution pump 54, through venting sterilizing filter 50, and back into the patient's peritoneal cavity.

Dialysate pump rotor 6 stops if any of three conditions occurs: two liters of regenerated dialysate has been pumped, fluid pressure sensor 41 (just upstream of venting sterilizing filter 50) senses above normal pressure, indicating that venting sterilizing filter 50 is becoming clogged, or fluid pressure sensor 41 senses below normal pressure, indicating that one of the membranes or fittings in venting sterilizing filter 50 is leaking.

During both halves of the operating cycle, the system monitors and controls the dialysate flow rate, and tracks the total amount of dialysate that has been pumped. The system also continuously measures the volume of spent dialysate in the fluid storage container.

At the start of the first cycle each evening, the control unit will vent trapped air from the sorbent cartridge, the tubing, and the venting sterilizing filter. This “air purge” cycle differs from the normal cycle as follows: at the start of the air purge cycle, glucose solution pump 53 activates a few cycles to flush air bubbles air out of its fittings, and air venting valve 44 opens for approximately 20 seconds. Also, a bolus of glucose solution is added to the dialysate stream to bring the dialysate's glucose concentration quickly up to the required level. Finally, an extra ½ liter of tap water is pumped from fluid storage container 57, to make up for the dead air volume in the sorbent cartridge and the cassette tubing.

Phrases Used in This Document: The numerical values and ranges in this document that specify mass, pH, volume, flow rate, etc., have been given as precisely as presently possible. However, unless otherwise indicated, all numbers and ranges specified in this document are to be understood as being modified by the term “about”. Ranges of values herein are intended to serve as a shorthand method of referring individually to each separate value falling within the range. Unless otherwise indicated herein, each individual value is incorporated into the specification as if it were individually recited herein.

The terms “a” and “an” and “the”, and similar referents used in this document are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified.

Preferred embodiments of this invention are described herein, including the best mode known to the inventor for carrying out the invention. Of course, upon reading the foregoing description, variations on those preferred embodiments will become apparent to those of ordinary skill in the art. This invention includes all modifications and equivalents of the subject matter recited in the claims as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A system for providing peritoneal dialysis, comprising:
a fluid storage container that can contain tap water and spent peritoneal dialysate, the fluid storage container having a fluid temperature sensor for sensing fluid temperature and a fluid volume sensor for sensing fluid volume;
at least one electric fluid heater for warming fluids in the fluid storage container;
a sorbent chemical cartridge containing Activated Carbon, fixed or unfixed Urease, Zirconium Phosphate, and Hydrous Zirconium Oxide, for absorbing toxins from spent dialysate;
a glucose solution cartridge containing concentrated glucose solution for regenerating spent dialysate;
a glucose solution pump for pumping concentrated glucose solution into spent dialysate;
an electrolytes solution cartridge containing concentrated electrolytes solution for regenerating spent dialysate;
an electrolytes solution pump for pumping concentrated electrolytes solution into spent dialysate;
a venting, sterilizing filter for sterilizing a dialysate, degassing a dialysate, and venting unwanted gasses from a dialysate;
an ammonia/ammonium sensor for detecting ammonia and/or ammonium dissolved in the dialysate;
a venting valve for venting entrapped air from a dialysate flow path;
a plurality of conduits for passage of dialysate connecting the fluid storage container, sorbent chemical cartridge, glucose solution cartridge, glucose solution pump, electrolytes solution cartridge, electrolytes solution pump, and venting, sterilizing filter;
a plurality of controllable fluid flow valves for selectably controlling the flow of dialysate through the conduits;
a plurality of controllable pumps for pumping dialysate through the conduits and controllable valves, and the controllable pumps including a dialysate pump for pumping dialysate from the fluid storage container to within a patient's peritoneal cavity and for pumping spent dialysate from a patient's peritoneal cavity into the fluid storage container;
a removable one-lumen dialysate tube for delivering generated or regenerated dialysate from the fluid storage container to a patient, and for withdrawing spent dialysate from a patient, and whereas the one-lumen dialysate tube is not part of a two or more lumen fluid loop system; and
a controller connected to the fluid temperature sensor, fluid volume sensor, electric fluid heater, ammonia/ammonium sensor, glucose solution pump, electrolytes solution pump, venting valve, controllable fluid flow valves, and controllable pumps, so that the controller controls the fluid temperature sensor, fluid volume sensor, electric fluid heater, ammonia/ammonium sensor, glucose solution pump, electrolytes solution pump, venting valve, controllable fluid flow valves, and controllable pumps to use tap water to generate peritoneal dialysate, to pump the generated peritoneal dialysate from the fluid storage container to a patent through the one-lumen dialysate tube, to confine the generated peritoneal dialysate within a patient's peritoneal cavity to produce spent peritoneal dialysate, to remove spent dialysate from a patient's peritoneal cavity through the one-lumen dialysate tube, to use spent dialysate to produce a regenerated peritoneal dialysate by passing spent dialysate through the sorbent cartridge and pumping concentrated electrolytes solution into the spent dialysate, to pump the regenerated dialysate from the fluid storage container into a patient through the one-lumen dialysate tube, and whereas the dialysate is pumped to a patient and thereafter removed from a patient in a tidal action such that the generated dialysate and regenerated dialysate are pumped to a patient via a single one-lumen dialysate tube, and at a later time, spent dialysate is removed from a patient via the same one-lumen dialysate tube.

2. The system of claim 1, wherein the dialysate pump is a peristaltic type.

3. The system of claim 1, wherein the fluid storage container is removable.

4. The system of claim 1, wherein the fluid storage container includes a removable lid, a self-sealing “break away” fluid connector, a fluid temperature sensor, and a fluid volume sensor.

5. The system of claim 1, further comprising a circuit board for a fluid volume sensor.

6. The system of claim 1, wherein the fluid heater is an electric heating pad.

7. The system of claim 1, wherein the dissolved ammonia sensor is a colorimetric type, including a light source, a light meter, and a circuit board;

8. The system of claim 1, wherein the glucose solution and the electrolytes solution pumps are driven by solenoids.

9. The system of claim 1, wherein the sterilizing filter also vents gases entrained in the dialysate.

10. The system of claim 1, wherein an electrolytes solution cartridge can contain any one of a plurality of electrolytes solutions, each solution having a different chemical formulation suitable for patients who are acidotic or alkylotic, or hypokalemic or hyperkalemic, or hypocalcemic or hypercalcemic, or hyponatremic or hypernatremic.

11. The system of claim 1, further comprising a sterilization port that uses ultraviolet light to sterilize a dialysate tube connector.

12. The system of claim 1, further comprising a circuit board (possibly an RFID type) and lead wires for reading the serial number chip on a cassette, a sorbent chemical cartridge, a glucose solution cartridge, and a sterilizing filter.

13. The system of claim 1, further comprising a unique serial number chip (possibly RFID type) on each of a cassette, a sorbent chemical cartridge, a glucose solution cartridge, and a sterilizing filter, for uniquely identifying each component.

14. The system of claim 1, further comprising a flash drive, a flash drive port, and a USB circuit board, for automatic storage of operational and alarm data for the system.

15. The system of claim 1, further comprising electrical connectors for allowing electronic communication between a cassette and a control unit, and electrical connectors for allowing electronic communication between a fluid storage container and a control unit.

16. The system of claim 1, further comprising an audio circuit board, a speaker and a selector switch that generate context-specific verbal operating instructions and verbal alarm messages, with the voice's language and gender being user selectable at all times.

17. The system of claim 1, further comprising a two axis inclinometer, for generating an alarm signal if the system becomes tilted out of horizontal orientation during operation.

18. The system of claim 1, further comprising docking bays for a glucose solution cartridge, a sorbent chemical cartridge, a sterilizing filter, and a removable fluid storage container, that all include self-sealing “break away” fluid connectors.

19. The system of claim 1, further comprising static positioning guides and a mechanical locking mechanism, for positioning and locking a cassette onto the top of the control unit.

20. The system of claim 1, further comprising a detachable carrying strap or handle.

21. The system of claim 1, wherein the sorbent chemical cartridge contains Activated Carbon, fixed or unfixed Urease, Zirconium Phosphate, and Hydrous Zirconium Oxide.

22. The system of claim 1, further comprising a control knob or button that sets the amount of glucose that the regenerated dialysate will contain, ranging from 0.0% to 6.0%.

23. The system of claim 1, wherein the controller includes software code that enables the device to perform a self rinse procedure.
